(19) United States
(12) Patent Application Publication
(10) Pub. No.: US 2003/0021816 A1
(43) Pub. Date: Jan. 30, 2003

(54) Compositions and methods for use against acne-induced inflammation and dermal matrix-degrading enzymes

(76) Inventors: Sewon Kang, Ann Arbor, MI (US); John J. Voorhees, Ann Arbor, MI (US); Gary J. Fisher, Ypsilanti, MI (US)

Correspondence Address: Bradley N. Ruben, PC, 463 First St., Suite 5A, Hoboken, NJ, 07030, US

(21) Appl. No.: 10/167,040

(22) Filed: Jun. 6, 2002

Related U.S. Application Data
(60) Provisional application No. 60/296,394, filed on Jun. 6, 2001.

(51) Int. Cl. A61K 38/13; A61K 31/7052; A61K 31/7048; A61K 31/4745; A61K 31/366; A61K 31/353

(52) U.S. Cl. 424/401; 514/11; 514/27; 514/147; 514/454; 514/28; 514/291

(57) ABSTRACT

Acne-affected skin has been found to be accompanied by the presence of matrix-degrading enzymes such as MMPs and neutrophil elastase, induction of neutrophils, and a reduction in procollagen biosynthesis. This invention treats scarring and inflammation accompanying acne by administering, topically or systemically, at least one of (i) an inhibitor of the matrix degrading enzymes and (ii) a cytokine inhibitor that alleviates inflammation and thus also alleviate neutrophil infiltration. Alleviating the matrix degradation and renormalizing procollagen biosynthesis allows for reduced inflammation and better natural repair of acne-affected skin. Inhibiting cytokines alleviates induction of MMPs in resident skin cells, and also alleviates inflammation with its concommitant induction of neutrophils from the blood stream bringing MMPs and elastase into the acne lesion. Dimishing the presence of matrix-degrading enzymes in the acne lesion reduces imperfect repair of the skin and thus decreases scarring in acne-affected skin.

This application is based on provisional application 60/296394, filed Jun. 6, 2001.

### TECHNICAL FIELD

This invention involves protecting human skin from some of the effects of acne, especially acne vulgaris, through the use of the topically and/or systemically applied non-retinoid and non-steriod compounds that diminish inflammation and matrix-degrading enzymes in acne-affected skin.

### BACKGROUND

Acne is a multifactorial disease, developing in the sebaceous follicles. At least one agent thought responsible is the anaerobe Propionibacterium acnes (P. acnes); in younger individuals, practically no P. acnes is found in the follicles of those without acne.

The disease of acne is characterized by a great variety of clinical lesions. Although one type of lesion may be predominant (typically the comedo), close observation usually reveals the presence of several types of lesions (comedones, pustules, papules, and/or nodules). The lesions can be either noninflammatory or, more typically, inflammatory. In addition to lesions, patients may have, as the result of lesions, scars of varying size. The fully developed, open comedo (ie., a plug of dried sebum in a skin pore) is not usually the site of inflammatory changes, unless it is traumatized by the patient. The developing microcomedo and the closed comedo are the major sites for the development of inflammatory lesions. Because the skin is always trying to repair itself, sheaths of cells will grow out from the epidermis (forming appendageal structures) in an attempt to encapsulate the inflammatory reaction. This encapsulation is often incomplete and further rupture of the lesion typically occurs, leading to multichanneled tracts as can be seen in many acne scars.

In general, there are four major principles presently governing the therapy of acne: (i) correction of the altered pattern of follicular keratinization; (ii) decrease sebaceous gland activity; (iii) decrease the follicular bacterial population (especially P. acnes) and inhibit the production of extracellular inflammatory products through the inhibition of these microorganisms; and (iv) produce an anti-inflammatory effect. The present treatments for acne following these principals typically include: vitamin A acid (retinoic acid), known for its comedolytic properties, administered topically (e.g., Retin-A® brand 0.025% all-trans retinoic acid cream) or systemically (e.g., Accutaneo brand 13-cis retinoic acid); an antibiotic administered systemically (e.g., tetracycline or one of its derivatives) or topically (e.g., benzoyl peroxide, erythromycin, clindamycin, azelaic acid); the use of other comedolytic agents such as salicylic acid; or the use of systemic anti-androgens such as cyproterone acetate and spironolactone (because androgens promote sebum production, and sebum has been found to be comedogenic and inflammatory), which may be administered in combination with an estrogen. Atrophy, the most feared side effect of topical glucocorticoids, is seen as an overall reduction in the dermal volume and occurs as early as one week after superpotent-steroid use. Systemic side effects of chronic glucocorticoid use include suppression of the hypothalamic-pituitary-adrenal (HPA) axis, Cushing's syndrome, glaucoma, and, in children, failure to thrive. (Children, especially infants and young children, are at higher risk for systemic side effects due to their greater surface-to-body ratio. They also may not metabolize corticosteroids as well as adults.) Withdrawal symptoms can appear after topical steriods have been used for a long period of time. Severe flaring may occur when isotretinoin (13-cis) therapy is started, and so concommitant use of a steriod, and suboptimal doses of isotretinoin, are often required at the start of therapy; additionally, retinoids generally are teratogenic (inhibiting organogenesis as opposed to being mutagenic).

The art has addressed inflammation and scarring caused by acne as a secondary benefit to the treatment of the disease; that is, if the acne is cured the factors causing scarring will be eliminated. There is otherwise no treatment directed at preventing scarring from acne. Neither is there presently any direct treatment for the inflammation accompanying acne. The conventional treatment acts to prevent further problems by alleviating the cause of the acne; for example, a patient is treated with tetracycline, an antiobiotic, in hopes of killing the P. acnes, and the death of the bacteria will effectively end the inflammation and future scarring. Much as antipyretics, analgesics, decongestants, and antihistamines have been developed to treat the symptoms of colds and upper respiratory infections (as opposed to antibiotics and antivirals to kill off the invading bacteria and viruses), there is a need for treatments diminishing if not preventing scarring and inflammation in acne.

As we have described in other applications, there are multiple factors leading to scarring and inflammation in acne. One of the factors leading to scarring is an inflammatory response, believed due to activation of TLRs (toll like receptors) due to LPS-like products as a result of the P. acnes infection in the follicle. This inflammatory aspect of acne has not been appreciated previously in the art. We have confirmed the existence of an inflammatory response mainly through increased levels of mRNA encoding for inflammatory cytokines.

### SUMMARY OF THE INVENTION

In brief, this invention provides a composition comprising an immunosuppressant compound, and preferably a second active ingredient selected from the group consisting of comedolytics, antibacterials, anti-inflammatories, retinoids, glucocorticoids, and mixtures thereof, and a dermatologically acceptable carrier. In one aspect, the use of an immunosuppressant compound alone should provide some relief of some of the symptoms of acne. Additional active ingredients, such as antibacterials and the like just mentioned can provide additional benefits.

Thus, in another aspect this invention provides a method for teating aspects of acne by using an immunosuppressant compound, preferably applied topically. This therapy can be conbined with other, more conventional therapies, such as antibiotics, glucocorticoids, retinoids, comedolytics, and anti-inflammatories, and combinations these more conventional therapies.

### BRIEF DESCRIPTION OF THE FIGURES

FIG. 1 is a histograph showing in vivo results of the existence of immunomodulatory compounds found in a biopsy of an acne lesion.

FIG. 2 is a cartoon explaining the various signalling pathways activated by acne to understand the possible mechanism by which the present invention can help to alleviate at least some symptoms of acne.

### DETAILED DESCRIPTION OF SPECIFIC EMBODIMENTS

The matrix of the skin (the dermal matrix), a structural framework that supports the cells and other structures in the skin, is comprised of collagen and elastin proteins for structural and dynamic (elastic) support.

Scarring of acne-affected skin has been known for a long time, and the typical treatment philosophy is that curing the acne will eliminate future scarring. As described in the Background section, it has also been known that acne includes bacterial infestation and an inflammatory reaction.

We have discovered that neutrophils (PMNs), immune cells that migrate to areas of injury, invade acne-affected skin, and release both a collagenase (MMP-8) and another protease (neutrophil elastase) that likely exacerbate scarring. Additionally, we have discovered that acne-affected skin has an elevated collagenase (MMP-1) level from resident skin cells that further exacerbates scarring. By inhibiting these dermal matrix-degrading enzymes, scarring of acne-affected skin can be lessened. Neutrophils circulate in the blood and therfore must be recruited by a signalling mechanism to induce their presence in the skin, facilitate their infiltration to the affected site, and enable their release of MMP-8 and elastase. Accordingly, impeding or disrupting the signalling which induces their presence in the skin and/or the activity of MMP-8 or elastase is likely to diminish the accompanying inflammation and the degradatory action of MMP-8 and/or elastase.

Matrix metalloproteinases (MMPs) are a family of enzymes that play a major role in physiological and pathological destruction of connective tissue, especially collagen. Various types of collagen and collagenases (types of MMPs) are known in this field, and a further description can be found in U.S. Pat. No. 5,837,224, and in our co-pending application Ser. No. 89,914, filed Jun. 3, 1998, the disclosures of which are incorporated herein by reference in their entirety for all purposes. Inhibitors of MMPs (e.g., direct inhibitors of the proteinase) and of molecular pathways (e.g., inhibitors of AP-1 and/or NF- B) that affect MMP expression are known in other fields and likewise are described in U.S. Pat. No. 5,837,224.

Macrolides are products of actinomycetes (soil bacteria) or semi-synthetic derivatives of them. They bind the 50S subunit of the bacterial ribosome thus inhibiting protein synthesis, and hence typically have an antibiotic or antimicrobial activity. Some, but not all, macrolides have immunosuppressant activity, which can be important in suppressing adverse reactions in acne.

Interleukin 2 (IL-2) is a lymphokine synthesized and secreted primarily by T helper lymphocytes that have been activated by stimulation with certain mitogens or by interaction of the T cell receptor complex with antigen/MHC complexes on the surfaces of antigen-presenting cells. The response of T helper cells to activation is induction of the expression of IL-2 and receptors for IL-2 and, subsequently, clonal expansion of antigen-specific T cells. Once a T-cell is activated by an antigen, a series of signals in the cytosol and nucleus, including the production of the transcription factor nuclear factor of activated T-cells (NFAT) by calcineurin, leads to the production of IL-2 and IL-2 high affinity surface receptors; hence a positive feedback can occur. By interfering in the signal pathway which leads to the produciton of IL-2, cyclosporin A (CsA) and FK-506 prevent a cell mediated immune responce. CsA and FK506 enter the cytosol and form complexes with the immunophilins cyclophilin and FK-binding protein-12 (FKBP-12) respectively. The protein-drug complexes prevent calcineurin, a calcium and calmodulin-dependent phosphotase, from producing NFAT, one transription factor of the tightly regulated IL-2 gene. Rapamycin like FK506 binds to FKBP-12 in the cytosol of T-cells, however, rapamycin inhibits the proliferation of T-cells through a different mechanism and in a different point in the proliferation of T-cells. The rapamycin-FKBP-12 complex interfers with the signal pathway at the point in which the T-cells have just entered the G1 phase. By blocking the IL-2 induced activation of p70 S6 kinase, the complex prevents the phosphorylation of ribosomal S6 thus preventing T-cell proliferation. NF-AT is also a transcription factor for other pro-inflammatory cytokines, such as IL-8 and TNFα.

Macrolides like FK506 (approved in the U.S. in various forms under the brands PROTOPIC and PROGRAF, and generically as Tacrolimus) form a complex which shields the active site of calcineurin, and inhibit IL-2 and the expression of IL-2 receptors. Macrolides like rapamycin inhibit the IL-2 based activation of T-cells. Another immunomodulatory macrolide is ascomycin, and derivatives thereof such as Ascomycin Macrolactam, ASM 981 (Novartis; also identified as SDZ-ASM-981 for Sandoz). These are rather strong compounds, typically used to prevent graft-host reactions in organ transplants, and may be toxic at such doses. E.g., McKane, W., “Treatment of calcineurin inhibitor toxicity by dose reduction plus introduction of mycophenolate mofetil,” Transplant Proc, 2001 Feb-Mar;33(1-2):1224-5.

Besides macrolides, other compounds have been found to inhibit the pathway from calcineurin and NF-AT to IL-2 and T-cells. For example, 4-(fluoromethyl)phenyl phosphate inhibits calcineurin binding. Born, TL, et al., “4-(Fluoromethyl)phenyl phosphate acts as a mechanism-based inhibitor of calcineurin,” J. Biol. Chem., 270, 25651-25655 (1995). Okadaic acid has an ID₅₀ value for calcineurin of about 4 μM, as does dibefurin, a fungal metabolite. Rusnak, F., and P. Mertz, “Calcineurin: Form and Function,” Phys. Rev., vol. 80, no. 4,1483-1521 (2000). Okadaic acid is a toxin was isolated from the sponge Halichondria okadai and is a complex lipophilic polyether readily soluble in many organic solvents, degrading in acid or base. A derivative of okadaic acid is Okadaol, 7-O-palmitoylokadaic acid (available from LC Laboratories division of Procyon Pharmaceuticals, Inc., Woburn, Mass.).

Macrolides are used in the treatment of pyoderma gangrenosum (“PG”), a destructive inflammatory skin decease of unknown etiology, yet often associated with various systemic diseases in which autoimmune mechanisms are known or suspected to occur. The inflammatory aspect of PG is known to be dominated by PMNs (polymorphic leukocytes; e.g., neutrophils). Yet PG is responsive to immunosuppressive macrolides such as FK509 and CsA, despite the fact that T cells, if present, are present in the minority.

Accordingly, in the context of this invention, provided is a method for treating acne by the topical administration of an immunomodulator, an immunosuppressant composition, and especially an inhibitor of calcineurin, NF-AT, or IL-2, or a combination thereof, such as by using any of the above compounds in a non-toxic, effective dose. As we have discovered, as described in the above-referenced patent applications, PMNs are present in acne lesions. Thus, as PG inflammation appears to be dominated by the presence of PMNs yet is responsive to immunosuppressant macrolides, acne inflammation should respond similarly. That is, even though these immunosuppressive compounds were investigated and developed primarily in relation to T cell activity, these compounds are useful in treating PG, a condition that, like we have found for acne, has an inflammatory component that is dominated by PMNs rather than T cells.

It is possible to determine whether a given compound is an inhibitor of calcineurin, NF-AT, or IL-2. For example, a population of cells is measured for its intrinsic IL-2 activity, the cells are then challenged with a substance and the IL-2 activity is measured again, and then the cells are treated with a candidate compound and challenged with the substance and the IL-2 activity is measured yet again and compared with the other two challenges to determine whether it is an inhibitor.

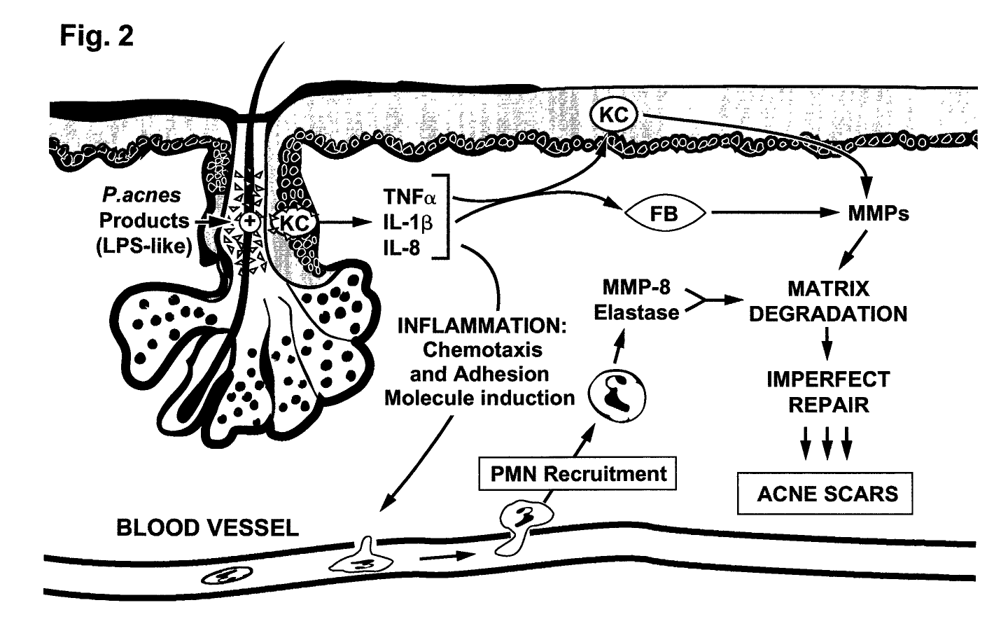

As shown in FIG. 2 of this application 1, IL-8 and TNFα are induced by signalling from TLRs due to the LPS-like acne products. Also, NF-AT is a transcription factor for these two cytokines. Accordingly, inhibition of the functioning of NF-AT is a treatment useful for reducing the inflammation in acne.

As mentioned above, the immunosuppressive macrolides, and compounds such as okadaic acid, are rather powerful, and thus have more toxicity issues when administered systemically/orally. In the context of acne, the epidermal barrier and inter-follicular epidermal barrier are compromised, hence topical delivery of these types of compounds is possible. For topical application, the compound is admixed with a compatible dermatologically acceptable carrier. For example, FK506 is available as PROTOPIC brand for the treatment of atopic dermatitis via topical administration. ASM 981 is currently available in cream formulation, and is being developed to treat atopic and irritant dermatitis. Of course, it is likely that in the future immunosuppressive macrolides will be developed or discovered that have fewer side effects than those presently available, and those safer macrolides could be administered orally in the treatment of acne if the benefits to treatment outweight the side effects from the drug. Topical delivery is facilitated by the use of a lipophilic molecule as the active ingredient and/or a lipophilic carrier. Often an emulsion or suspension can be used to deliver compounds that do have the desire lipophilicity.

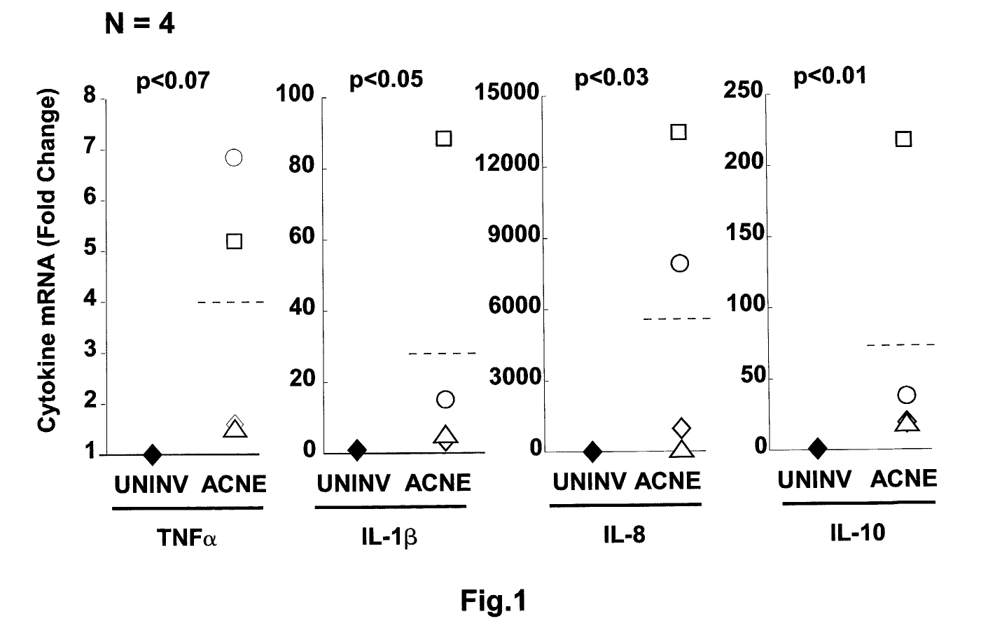

FIG. 1 shows the difference in the amount of mRNA encoding for the inflammatory cytokines mentioned above (TNF, IL-1, IL-8, and IL-10) between uninvolved and acne-affected skin. Each of the individuals (human volunteers having given informed consent) is represented by a differently-shaped data point, and the mean value is shown as the horizontal dashed line. As with collagen-degrading enzymes, the amounts of mRNA encoding the cytokines increased from ininvolved to acne-affected skin: TNF was about four times higher, IL-1 was over 25 times higher, IL-8 was over 5000 times higher, and IL-10 was about 75 times higher. These data confirm that increased cytokine concentrations (inferred from an increase in their mRNA levels) are present in acne lesions, and thus the scarring and collagen degradation due to acne can be treated with a combination of MMP inhibitors and cytokine inhibitors.

While not desirous of being constrained to a particular theory, the possible mechanism by which this invention functions is depicted in the cartoon of FIG. 2. On the left side of FIG. 2 a hair follicle infected with P. acnes is shown. These bacteria release LPS (lipopolysaccharide)-like compounds which are sensed by keratinocytes (KC) (triangles in FIG. 2). (B R Vowels, S Yang, and J J Leyden, “Induction of proinflammatory cytokines by a soluble factor of Propionibacterium acnes: implications for chronic inflammatory acne, Infect. Immun. 1995 63: 3158-3165; the disclosure of which is incorporated herein by reference). The toll-like receptor (TLR) family includes LPS receptors, and those in the keratinocytes are activated by LPS-like products from P. acnes. Activation of the TLRs causes NF- B to enter the cell nucleus of keratinocyes. The keratinocytes are thus induced to release chemotactic factors, especially cytokines (IL-1, IL-8, IL-10, TNF). These factors activate the AP-1 and NF-B pathways, and NF-B activates more IL-1 and TNF (a cyclical process; see FIG. 1 in our prior patent U.S. Pat. No. 5,837,224 on photoaging due to UV radiation, the disclosure of which is incorporated herein by reference). The release of these factors causes inflammation, including the recruitment of neutrophils (PMNs; i.e., polymorphonuclear leukocytes) from the blood supply to the acne lesion; MMP-8 and elastase are preformed in the neutrophils and so their presence in skin is due to their presence in neutrophils. As shown in this cartoon, the cytokines also effect other keratinocytes and fibroblasts (FB), which are resident in the skin, to generate MMPs. The induction of matrix-degrading enzymes due to the presence of acne, and the continual repair of the damage they do, leads to imperfect repair of the skin. Thus, elimination of the enzymes that degrade the dermal matrix reduces imperfect repair of the skin, and so lessens scarring. Collagenase expression in acne-affected skin occurs in the dermis. Accordingly, a preferred composition includes indirect inhibitors of matrix degrading enzymes, such as glucocorticoids that block recruitment of neutrophils and other inflammatory immune cells, optionally retinoids that inhibit MMPs in resident skin cells, and direct inhibitors of these enzymes, such as serpine (a serine protease inhibitor analogous to TIMP), all preferably in combination with at least one compound for treating acne (e.g., benzoyl peroxide or tetracycline). While retinoids and antibacterials are commonly used to treat acne, they have not been used in combination with non-retinoid MMP inhibitors, elastase inhibitors, and/or inhibitors of the PNM recruitment pathway leading to degradation of the dermal matrix.

The foregoing description is meant to be illustrative and not limiting. Various changes, modifications, and additions may become apparent to the skilled artisan upon a perusal of this specification, and such are meant to be within the scope and spirit of the invention as defined by the claims.

what is claimed is:

1. A composition comprising: an immunosuppressant compound; a second active ingredient selected from the group consisting of comedolytics, antibacterials, anti-inflammatories, retinoids, glucocorticoids, and mixtures thereof, and a dermatologically acceptable carrier.

2. A method for treating acne, comprising administering to a patient in need thereof a non-toxic, immunosuppressive effective amount of an immunosuppressant compound.

3. The method of claim 2, wherein the compound is applied topically and is administered in a dermatologically suitable carrier.

4. The method of claim 2, wherein the immunosuppressant compound is selected from the group consisting of macrolides, 4-(fluoromethyl)phenyl phosphate, mycophenolate mofetil, cyclosporins, and okadaic acid and derivatives thereof.

5. The method of claim 2, wherein the immunosuppressant compound is selected from the group consisting of macrolides, 4-(fluoromethyl)phenyl phosphate, mycophenolate mofetil, cyclosporins, and okadaic acid and derivatives thereof.

6. A method for treating acne, comprising administering to a patient in need thereof a non-toxic, effective amount of an inhibitor of NF-AT.

7. The method of claim 6, wherein the administration is topically and the inhibitor is provided in a dermatologically suitable carrier.

8. Use of an immunomosuppressant in the treatment of acne.
